(19) United States
(12) Patent Application Publication
(10) Pub. No.: US 2017/0367502 A1
(43) Pub. Date: Dec. 28, 2017

(54) Management System for First Aid Supplies

(71) Applicant: Acme United Corporation, Fairfield, CT (US)

(72) Inventor: Dan Bittinger, Oregon City, OR (US)

(21) Appl. No.: 15/535,573

(22) PCT Filed: Dec. 18, 2015
(86) PCT No.: PCT/US15/66667

§ 371 Date: Jun. 13, 2017

Related U.S. Application Data
(60) Provisional application No. 62/096,059, filed on Dec. 23, 2014.

(51) Int. Cl. A47F 5/08; A61F 17/00

(52) U.S. Cl. CPC A47F 5/0807; A61F 17/00

(57) ABSTRACT

A supply management system for first aid supplies employs an organizing board. The organizing board has a multiplicity of forward axially projecting pegs which are configured in a generally rectangular grid. A plurality of first aid items are each contained in a box-like container having a rear mounting panel. The mounting panel has a matrix of structures defining apertures corresponding in spacing to selected portions of the grid of pegs. Each first aid container is mountable to the board in a stable position whereby selected pegs are received in apertures of the mounting panel and the panel is disposed forwardly from the board. The structures are deformable and have a generally circular central portion with a plurality of radial perforations which form tabs. The organizing board is preferably placed against the rear of a first aid cabinet. The organizing board and the matrix of apertures ensure compatibility of first aid replacement products.

### BACKGROUND

This disclosure relates generally to first aid supplies and first aid kits. More particularly, this disclosure relates to systems for ensuring the proper replacement of first aid supplies.

First aid kits are commonplace and are provided in numerous sizes and adapted for numerous applications. A recurring problem is that after initial acquisition of a first aid kit, the replacement of spent supplies with proper replacement supplies is often haphazard and the replacement first aid product may differ from the original product in significant respects. When the replaced product is employed during an emergency situation, the effectiveness of the treatment using the replaced first aid product can be compromised. Naturally, it is also highly desirable for any first aid kit that the first aid supplies, including the replacement supplies, be arranged in an organized fashion to provide ready access and identification.

### SUMMARY

Briefly stated, a supply management system for a plurality of items each disposed in a box-like container employs an organizing board. The organizing board has a multiplicity of forwardly axially projecting pegs configured in a generally rectangular grid. Various items are each disposed in a box-like container. The container has a rear mounting panel with a matrix of structures defining apertures corresponding in spacing to selected portions of the grid of pegs. Each container is mountable to the board in a stable position whereby selected pegs are received in apertures of the mounting panel and the container is disposed forwardly from the board.

The pegs are preferably substantially identical, but may assume different forms. In one embodiment, the pegs have a generally tapered point configuration. In another embodiment, the pegs form a notch or recess with a retaining edge. The pegs preferably axially extend from a planar base surface and terminate at a uniform distance from the surface. The structures are deformable and preferably have a generally circular center portion with a plurality of radial projections which, upon mounting, are deformable and engage against a peg. In one embodiment, at least one aperture is enlarged in comparison to other apertures.

The supply management system may comprise a housing having a rear side and a front cover which is displaceable to provide access to the housing. An organizing board is disposed adjacent the rear side. The organizing board has a multiplicity of forwardly axially projecting pegs configured in a generally rectangular grid. Various items are each disposed in a container. The container has a rear mounting panel with a matrix of structures defining apertures corresponding in spacing to selected portions of the grid of pegs. Each of the containers is mountable to the board in a stable position whereby selected pegs are received in apertures of the mounting panel and the containers disposed forwardly from the board are encloseable in the housing. The pegs are preferably substantially identical. The pegs may have a generally tapered pointed configuration. The pegs may form at least one notch or recess with a retaining edge.

In one embodiment, the housing has a plurality of shelves and the organizing board comprises a plurality of strips disposed between the shelves. The structures are deformable and have a generally central portion with a plurality of radial projections.

In one embodiment, the supply management system employs an organizing board which substantially covers the rear panel. The organizing board may comprise a plurality of bases with interlocking edges. In another embodiment a supply management system for a plurality of first aid items comprises a cabinet having a rear side and a front side with a cover which is displaceable to provide access to the cabinet. An organizing board is disposed adjacent the rear side and has a multiplicity of forwardly axially projecting pegs configured in a generally rectangular grid. A plurality of first aid items are each disposed in a box-like container. The container has a rear mounting panel with a matrix of deformable structures defining apertures corresponding in spacing to selected portions of the grid of pegs. Each container is mountable to the board in a stable position whereby selected pegs are received in apertures of the mounting panel and the containers disposed forwardly from the board and enclosable in the cabinet.

At least some of the pegs have a generally tapered pointed configuration. At least some of the pegs form a recess with a retaining edge. The deformable structures have a plurality of radial perforations which form tabs which are bendable to engage a received peg. The organizing board substantially covers the rear side of the cabinet.

### BRIEF DESCRIPTION OF THE DRAWINGS

FIG. 1 is a perspective view of a representative grid together with a representative first aid package for the management system for first aid supplies;

FIG. 2 is a front perspective view of an open first aid kit which implements the management system for first aid supplies;

FIG. 3 is a perspective view of another open first aid kit implementing the management system for first aid supplies;

FIG. 4 is a representative perspective view of another embodiment of the management system for first aid supplies;

FIG. 5 is a perspective view of one embodiment of a grid employed in the management system;

FIG. 6 is a diagrammatic perspective view of a portion of a grid employed in the management system;

FIG. 7 is a diagrammatic layout view of a first aid package for the management system;

FIG. 8 is a front diagrammatic view of a pair of panels in an intermediate stage further illustrating a side-by-side disposition for the management system;

FIG. 9 is a rear elevational view of a rear panel of a package for the management system;

FIG. 9A is an enlarged view of a portion of the panel of FIG. 9;

FIG. 10 is an enlarged diagrammatic layout view of another package for the management system;

FIG. 11 is an enlarged sectional view, partly in phantom, of a grid for the management system;

FIG. 11A is an enlarged diagrammatic view, partly in phantom, of a portion of the grid of FIG. 11 designated by the letter A;

FIG. 11B is a perspective view of the grid of FIG. 11;

FIG. 12 is a sectional view, partly in phantom, of yet another embodiment of a grid for the management system;

FIG. 12A is an enlarged diagrammatic sectional view, partly in phantom, of a portion of the grid of FIG. 12 designated by the letter B;

FIG. 12B is a perspective view of the grid of FIG. 12;

FIG. 13 is an enlarged sectional view, partly in phantom, of another embodiment of a grid of the management system;

FIG. 13A is an enlarged diagrammatic sectional view, partly in phantom, of the grid of FIG. 13 designated by the letter C;

FIG. 13B is a perspective view of the grid of FIG. 13;

FIG. 14 is a schematic view, partly in phantom and partly annotated, illustrating four first aid containers overlaying a single representative grid;

FIG. 15 is a perspective view of another embodiment of a grid; and

FIG. 16 is a frontal annotated view of a single grid with mounting holes.

### DETAILED DESCRIPTION

With reference to the drawings, a management system for first aid supplies employs an organizing board which defines a grid 10. The grid 10 interfaces with a multiplicity of first aid supplies in the form of packages each containing box-like containers 12 containing a first aid product (not illustrated). Each container 12 preferably has a thin cardboard construction which is folded and joined to form a rectangular box. The containers 12 may be constructed in different dimensions, house various supplies and have corresponding exterior identifying information. The various containers are designated by 12 and a distinguishing alphabetic letter to indicate the noted differences.

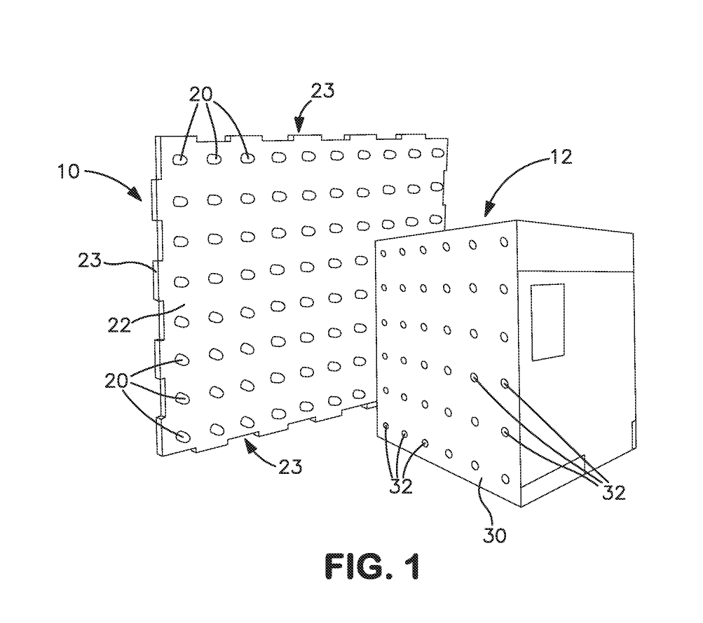

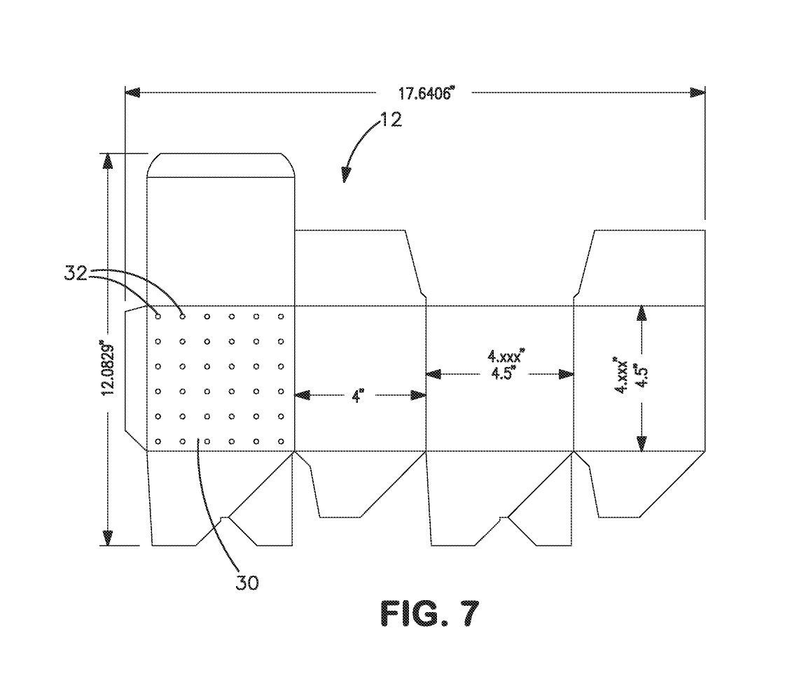

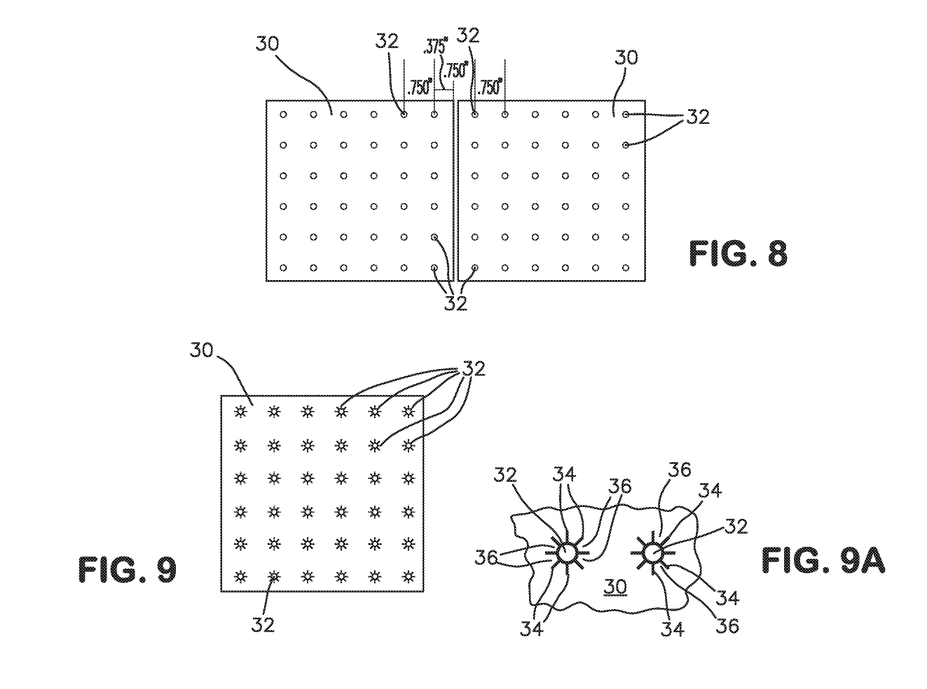

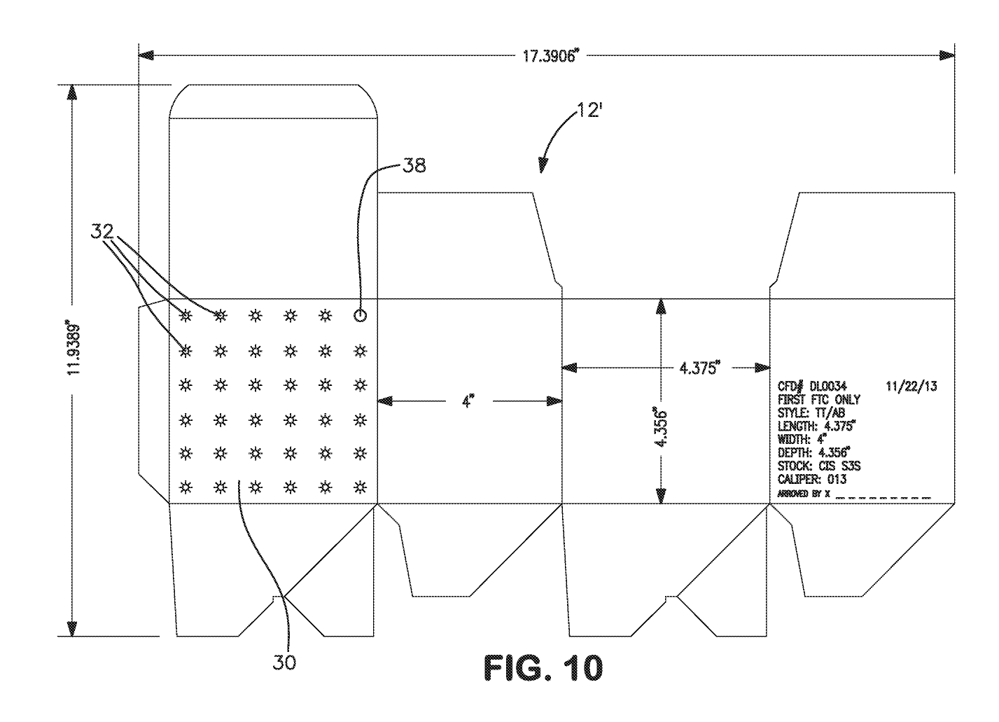

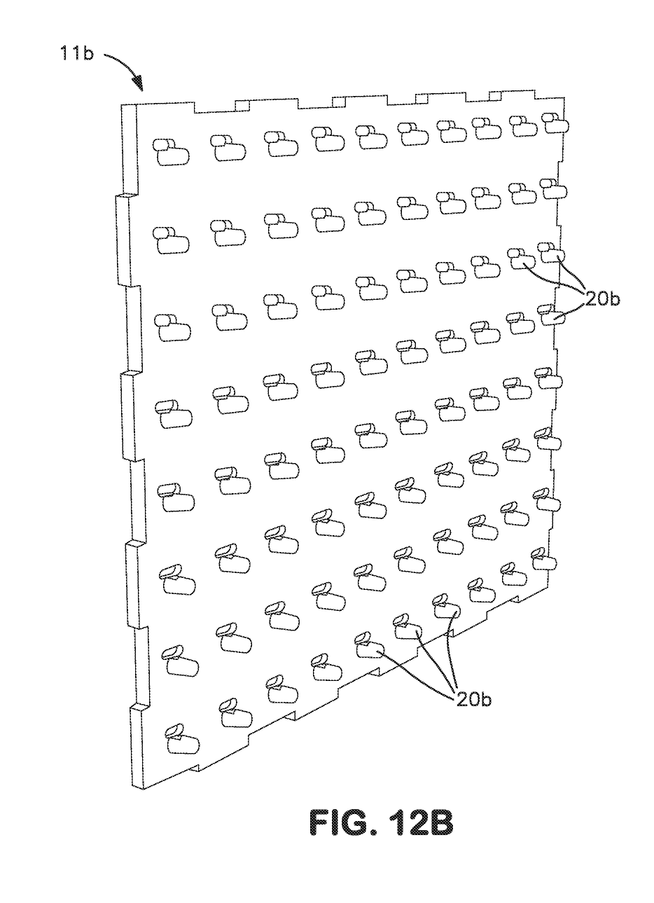

The grid 10 comprises a rectangular matrix of pegs 20 which axially project from a planar receiving surface 22. The pegs 20 define a central axis and are equidistantly rectilinearly spaced on the peg axes throughout the grid. The grid 10 may assume various dimensions and shapes, such as grid 10 of FIG. 5 or FIG. 6. Each grid has a base 24 (FIGS. 11-13) with a receiving surface 22 which is preferably planar and, in some embodiments, terminates in interlocking edges 23 as illustrated, for example, in FIG. 1. Alternative peg spacing relationships, such as greater spacing distances along one grid axis, are also possible.

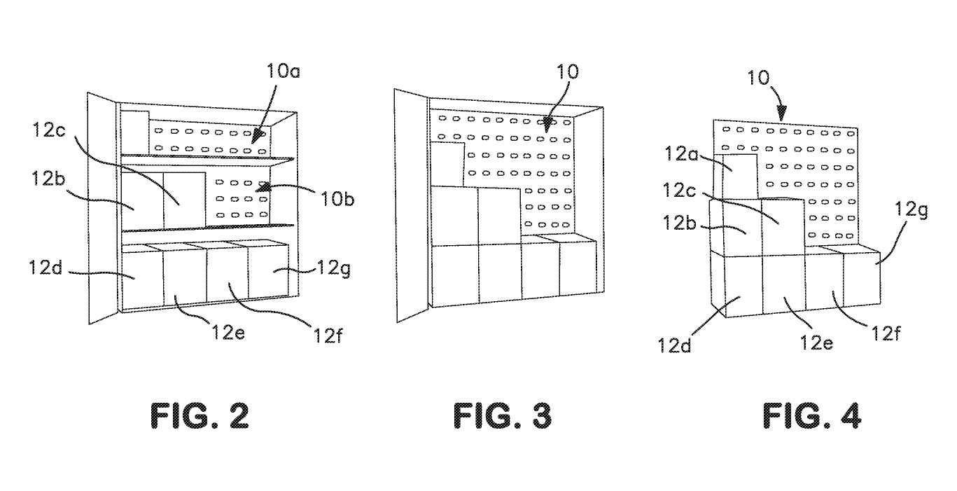

In some embodiments, the grid is configured in strips, such as grids 10a and 10b illustrated in FIG. 2. In one embodiment (not illustrated), the grid base and/or surface 22 has perforations which allow grid portions to be broken off and removed to tailor the grid to a given dimensional constraint. Each grid 10, 10a, 10b is preferably a plastic molded component.

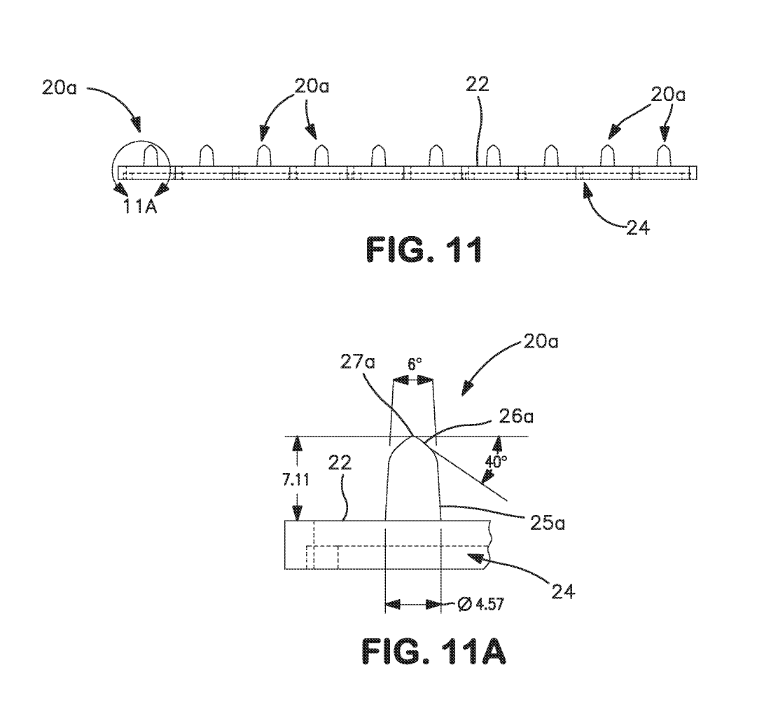

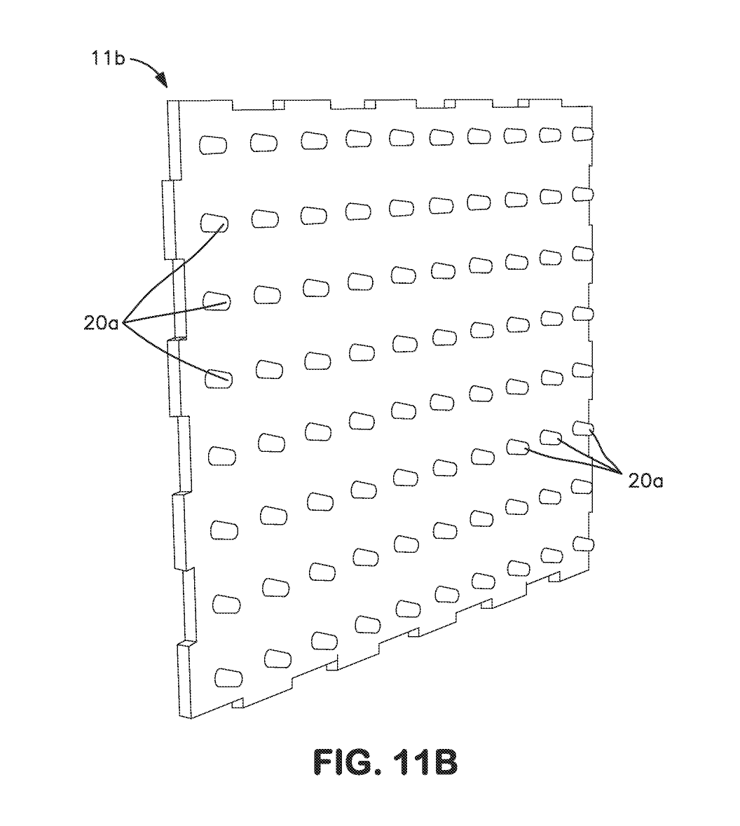

Container 12 includes a back panel 30 which is configured with a rectangular matrix of apertures 32 equidistantly spaced on-center so as to have spacing identical to that of the on-center spacing of the pegs 20 of the grid. The apertures 32 are preferably configured in an initial quasi-star-like configuration, as best illustrated in FIGS. 9 and 9A, wherein each aperture 32 has a plurality of radial or spoke-like perforations 34. The perforations 34 facilitate the formation of retaining tabs 36. The remaining portions of the container 12 are generally configured both in shape and exterior appearance in a manner similar to conventional first aid supply packaging. It should be appreciated, as best illustrated in FIG. 8, that the distance on-center from an outermost aperture to the edge of the container is slightly less than ½ the distance between the on-center distance of the pegs 20 to allow for adjacent side-by-side positioning of the containers.

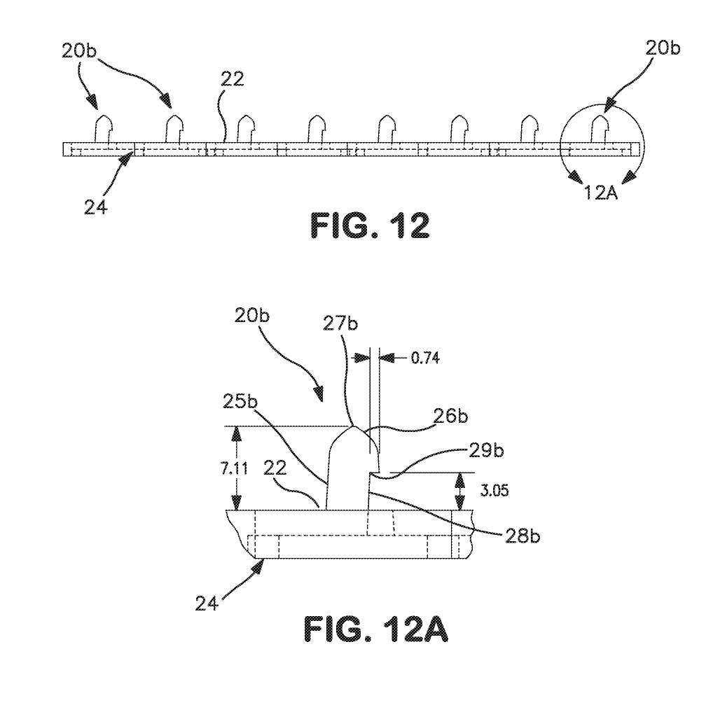

In one embodiment of the container 12′ illustrated in a preassembled layout form in FIG. 10, a corner aperture 38 is configured in a slightly enlarged circle to aid in the initial alignment of the container with a selected peg of the grid.

It will be appreciated that the rear panel 30 of the package or container with the matrix of apertures 32 is dimensioned and spaced so that it corresponds to the dimensioning and spacing of the pegs 20. Consequently, the first aid package can be selectively positioned and pushed against the grid so that the pegs 20 enter into the apertures 32 in a snug-like reception. The pegs 20 are shaped and dimensioned to force the portions 36 between the perforations 34 to fold inwardly in tab-like fashion and retentively engage against the projecting pegs 20. The receiving surface 22 and the rear panel 30 preferably engage in surface-to-surface relationship.

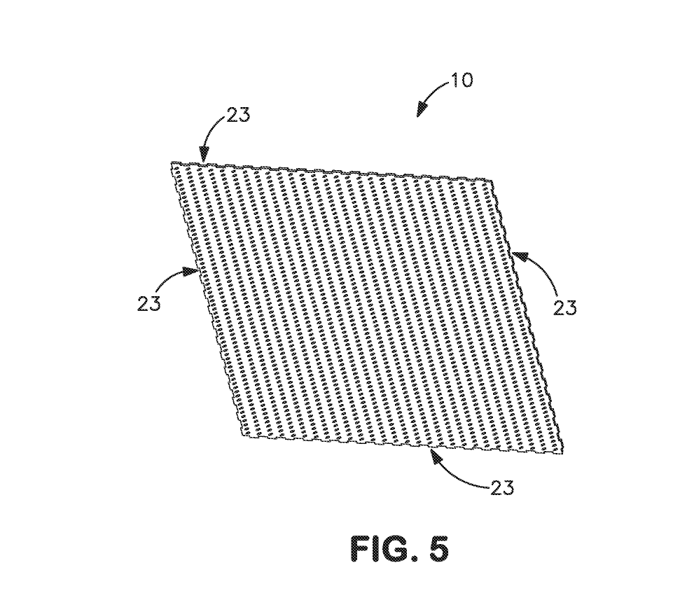

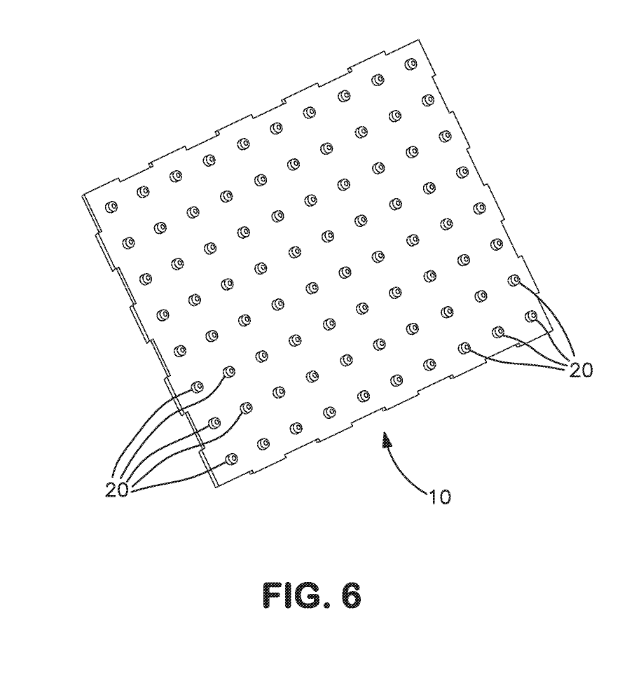

The spacing of the apertures 32 closest to the container edge allows the containers to be mounted to the grid in essentially side-by-side disposition, such as illustrated in FIGS. 2-4. In addition, the bottom and top spacing of the apertures from the edges of the container allow the containers to be disposed in adjacent vertical dispositions, such as illustrated in FIGS. 3 and 4.

For a given grid, it is preferred, although not required, that the pegs have identical configurations and extend the same axial distance from the planar mounting surface 22 of the base 24 of the grid. FIGS. 11-13B show three different peg configurations 20a, 20b and 20c, respectively. Analogous structures are designated by the letters a, b and c, respectively.

For peg 20a, as best illustrated in FIG. 11A, the peg 20a has a slightly tapered side 25a and a contoured tapered point 26a terminating in a small flat tip 27a. A grid 11a incorporating pegs 20a is illustrated in FIG. 11B.

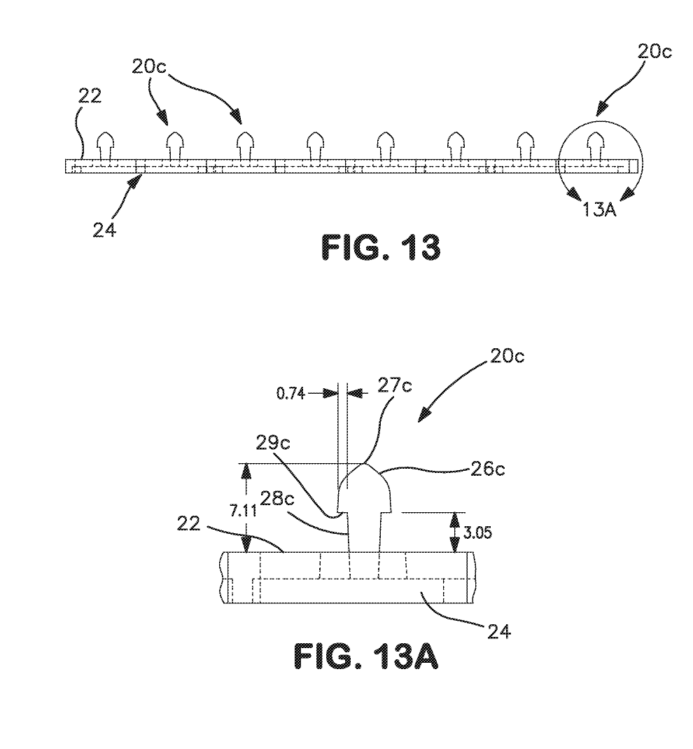

Peg 20b, as best illustrated in FIG. 12A, has a slightly tapered cylindrical portion 25b and a contoured tapered point 26b terminating in a small flat tip 27b. In addition, a notch or recess 28b is formed around a lower portion of the peg. The recess 28b is upwardly defined by an edge 29b which engages over the received back panel portions 36 adjacent the aperture 32 to implement a retaining engagement. A grid 11b incorporating pegs 20b is illustrated in FIG. 12B.

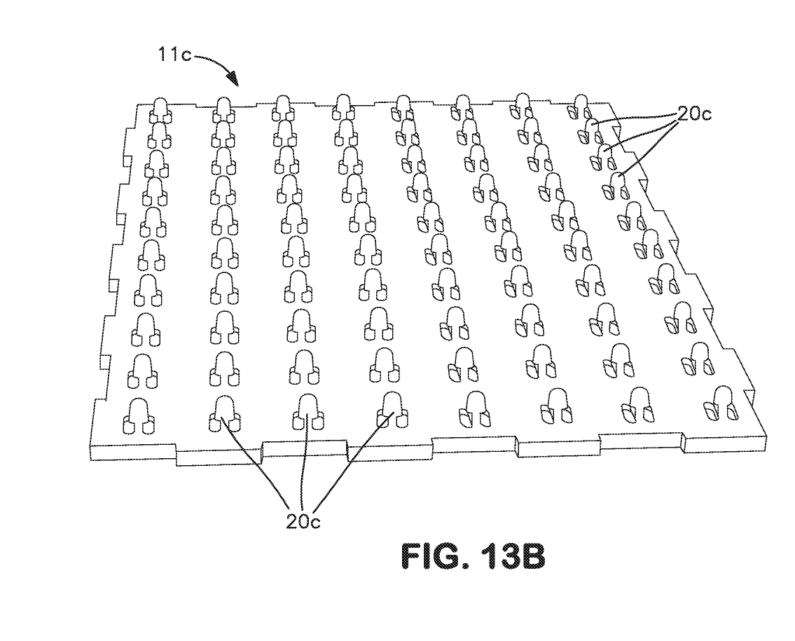

Peg 20c, as best illustrated in FIG. 13A, has a dual notched configuration with a contoured point 26c and a small flat tip 27c. The lower circumference forms a slightly tapered pair of notches or recesses 28c each defined by an upper edge 29c which forms a retaining surface when the container is moved against the peg and the surrounding portions of the peg slightly engage against the retaining folded edge portions 36. A grid 11c incorporating pegs 20c is illustrated in FIG. 13B.

In an alternative configuration (not illustrated), the peg may have a circumferential recess. Naturally, other forms of the pegs may be provided. It is preferred that the apertures and the pegs be dimensioned so that as the panel is forced against the plane of the grid, the outer portions defining the aperture slightly flex or deform to engage against the pegs. In some instances, the outer edges of the flexed portions 36 of the apertures are retained against the retaining edges 29b and 29c of the pegs.

The grids 10, 10A, 10B, 11A, 11B and 11C may be configured in numerous dimensional constraints conforming to various sizes of the first aid kit case. The grid is mounted against the back wall of the kit. In the embodiment illustrated in FIG. 2, the grids 10a and 10b are formed in strips and placed against the back of the kit between various shelves.

In the configuration of FIG. 3, the grid 10 conforms to the entire inside surface of the first aid kit case and provides a mounting board to allow the various supply containers to be forced against the pegs to thereby secure the containers of the various first aid supplies in a fixed organized orientation within the case.

The grid illustrated in FIG. 4 shows the various first aid containers mounted to a standalone grid without reference to any cover or side enclosing structures.

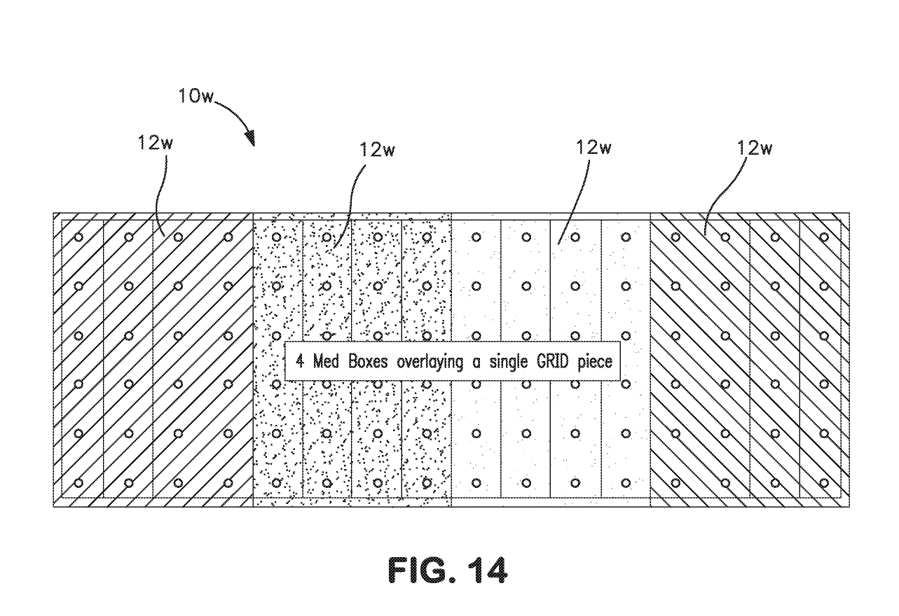

With reference to FIG. 14, a single grid 10w is illustrated which mounts four containers 12w in side-by-side disposition. It will be appreciated that the drawing is a schematic drawing with portions in phantom to show the peg/aperture relationships.

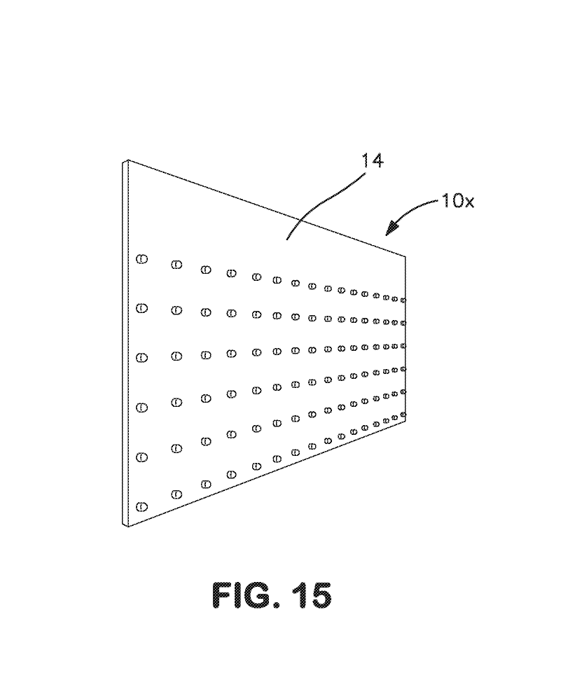

With reference to FIG. 15, a grid 10x essentially constitutes a wall board which may be constructed in numerous sizes. A header 14, which may include a label (not illustrated), is disposed at the central top portion of the grid. The grid 10x may be mountable with two-sided adhesive (not shown) or with a whole screw arrangement (not illustrated).

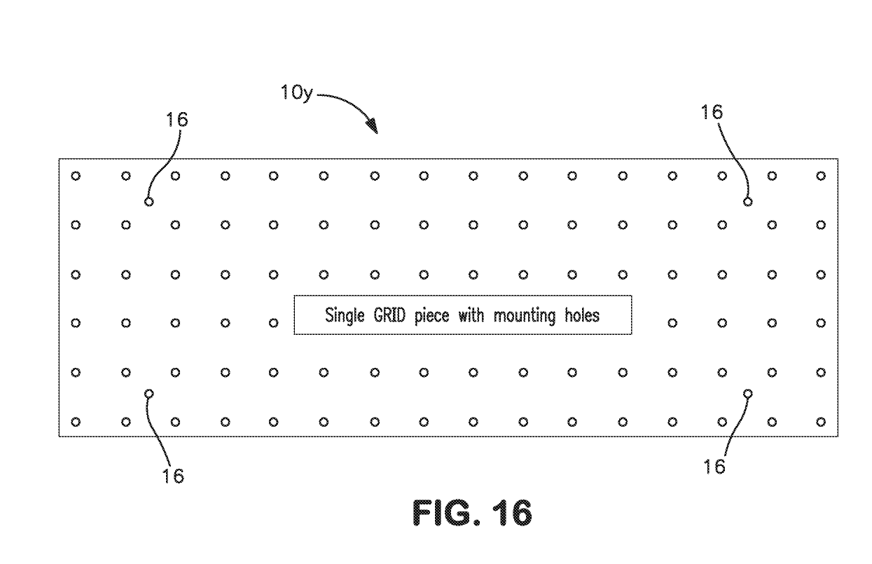

With reference to FIG. 16, a single grid 10y, which shows a multiplicity of pegs, includes mounting holes 16 for receiving a fastener (not illustrated) for mounting the grid to a wall, panel, an interior portion of a cabinet or otherwise.

The management system provided by the organizing board which may comprise multiple grid components and the rear mounting panel with the matrix of structures defining apertures of the container for the first aid supplies is readily adaptable for retrofitting existing first aid kits and cabinets to ensure orderly organization of the first aid supplies and proper replacement of the supplies when required. The flexibility of the organizing board, which can be composed of separate components and can be assembled and mounted as required for a given first aid cabinet, also provides a high degree of flexibility.

While preferred embodiments of the foregoing have been set forth for purposes of illustration, the foregoing description should not be deemed a limitation of the invention herein. Accordingly, various modifications, adaptations and alternatives may occur to one skilled in the art without departing from the spirit and the scope of the present invention.

1. A supply management system for a plurality of items each disposed in a box-like container comprising:
an organizing board having a multiplicity of forwardly axially projecting pegs configured in a generally rectangular grid;
a plurality of items each disposed in a box-like container having a rear mounting panel having a matrix of structures defining a grid of apertures corresponding in spacing to selected portions of the grid of pegs;
wherein each said container is mountable to said board in a stable position whereby selected pegs are received in apertures of said mounting panel and said container is disposed forwardly from said board.

2. The supply management system of claim 1 wherein said pegs are substantially identical.

3. The supply management system of claim 1 wherein said pegs have a generally tapered pointed configuration.

4. The supply management system of claim 1 wherein said pegs form a recess with a retaining edge.

5. The supply management system of claim 1 wherein said pegs axially extend from a planar base surface and terminate at a uniform distance.

6. The supply management system of claim 1 wherein said structures are deformable and have a generally circular central portion with a plurality of radial perforations which are deformable upon mounting to the organizing board.

7. The supply system of claim 1 wherein at least one aperture is enlarged in comparison to other apertures.

8. A supply management system for a plurality of items each disposed in a container comprising:
a housing having a rear side and a front side which is displaceable to provide access to said housing;
an organizing board disposed adjacent said rear side having a multiplicity of forwardly axially projecting pegs configured in a grid;
a plurality of items each disposed in a container having a rear mounting panel comprising a matrix of structures defining a grid of apertures corresponding in spacing to selected portions of the grid of pegs;
wherein each said container is mountable to said board in a stable position whereby selected pegs are closely received in apertures of said mounting panel and said container is disposed forwardly from said board and encloseable in said housing.

9. The supply management system of claim 8 wherein said pegs are substantially identical.

10. The supply management system of claim 8 wherein said pegs have a generally tapered pointed configuration.

11. The supply management system of claim 8 wherein said pegs form a recess with a retaining edge.

12. The supply management system of claim 8 wherein said housing has a plurality of shelves and said organizing board comprises a plurality of strips disposed between said shelves.

13. The supply management system of claim 8 wherein said structures are deformable and have a generally circular central portion with a plurality of radial perforations.

14. The supply management system of claim 8 wherein said organizing board substantially covers said rear side.

15. The supply management system of claim 8 wherein said organizing board comprises a plurality of bases with interlocking edges.

16. A supply management system for a plurality of first aid items each disposed in a box-like container comprising:
a cabinet having a rear side and a front side comprising a cover which is displaceable to provide access to said housing;
an organizing board disposed adjacent said rear side having a multiplicity of forwardly axially projecting pegs configured in a generally rectangular grid;
a plurality of first aid items each disposed in a box-like container having a rear mounting panel having a matrix of deformable structures defining a grid of apertures corresponding in spacing to selected portions of the grid of pegs;
wherein each said container is mountable to said board in a stable position whereby selected pegs are closely received in apertures of said mounting panel and said container is disposed forwardly from said board and encloseable in said cabinet.

17. The supply management system of claim 16 wherein at least some of said pegs have a generally tapered pointed configuration.

18. The supply management system of claim 16 wherein at least some of said pegs form a recess with a retaining edge.

19. The supply management system of claim 16 wherein said deformable structures each have a plurality of radial perforations which are bendable to engage a received peg.

20. The supply management system of claim 16 wherein said organizing board substantially covers said rear panel.
